(19) United States
(12) Patent Application Publication
(10) Pub. No.: US 2017/0340550 A1
(43) Pub. Date: Nov. 30, 2017

(54) AGENTS AND METHODS FOR THE TEMPORARY SHAPING OF KERATIN-CONTAINING FIBERS

(71) Applicant: HENKEL AG & CO. KGAA, Duesseldorf (DE)

(72) Inventors: Thomas FOERSTER, Duesseldorf (DE); Rolf BAYERSDOERFER, Hamburg (DE); Thorsten KNAPPE, Schenefeld (DE)

(73) Assignee: Henkel AG & Co. KGaA, Duesseldorf (DE)

(21) Appl. No.: 15/534,235

(22) PCT Filed: Nov. 2, 2015
(86) PCT No.: PCT/EP2015/075398

§ 371 Date: Jun. 8, 2017

(30) Foreign Application Priority Data
Dec. 10, 2014 (DE) 10 2014 225 443.4

(51) Int. Cl. A61K 8/81; B05B 1/30; A45D 7/04; A45D 34/04; A61K 8/41; A61K 8/34; B05B 9/00; A61Q 5/06

(52) U.S. Cl. CPC A61K 8/8182; B05B 9/005; B05B 9/002; B05B 1/3006; A61Q 5/06; A45D 34/04; A61K 8/8152; A61K 8/416; A61K 8/34; A45D 7/04; A61K 2800/87

(57) ABSTRACT

The disclosure relates to a cosmetic product, comprising a) a cosmetic preparation, containing, in relation to its total weight, a1) from about 60 to about 98 wt. % of at least one polar solvent; a2) from about 0.5 to about 20 wt. % of at least one copolymer from i) vinylpyrrolidone ii) vinyl acetate iii) and optionally other monomers; b) a device for flash evaporation of the cosmetic preparation a), and to a method using corresponding products and to the use of the cosmetic preparation a) as process material in a device for flash evaporation.

### CROSS-REFERENCE TO RELATED APPLICATION

This application is a U.S. National Stage entry under 35 U.S.C. §371 based on International Application No. PCT/EP2015/075398, filed Nov. 2, 2015 which was published under PCT Article 21(2) and which claims priority to Application no. 10 2014 225 443.4, filed Dec. 10, 2014, which are all hereby incorporated in their entirety by reference.

### TECHNICAL FIELD

The application relates to the technical field of the temporary shaping of keratin-containing fibers, particularly human hair. The object of the application is specific cosmetic formulations for the hair which are suitable for application to keratin-containing fibers using a flash evaporation method. The use of these cosmetic formulations for the hair in devices for flash evaporation and methods for the temporary reshaping of keratin-containing fibers represent further objects of the present application.

### BACKGROUND

Nowadays, an attractive hairstyle is generally considered to be an essential part of a well-groomed appearance. At the same time, modern fashion trends are constantly reviving the popularity of hairstyles which for many types of hair can only be created or maintained for longer periods, up to several days, by using strengthening ingredients. Therefore, hair treatment agents that provide permanent or temporary reshaping of the hair have an important role to play. Whereas in permanent reshaping the chemical structure of the keratin-containing fibre is modified by reduction and oxidation, such modifications of the chemical structure are not used in temporary reshaping. Corresponding agents for temporary reshaping usually contain synthetic polymers and/or waxes as strengthening agents.

The most important property of an agent for temporary reshaping of keratin-containing fibers, which will be called ‘styling agents’ in the following, consists in giving the strongest possible hold to the treated fibers in the newly styled form—i.e. a form impressed on the fibers. If the keratin-containing fibers are human hair, one also speaks of a strong hairstyle hold or high degree of hold of the styling agent. Styling hold is essentially determined by the type and quantity of strengthening ingredients used, although the other components of the styling agent and the application form may also be influencing factors.

In the field of the temporary reshaping of keratin-containing fibers, sprayed application of corresponding cosmetic preparations is very important, wherein the preparations are generally applied as pump sprays or aerosol sprays. For this purpose, the cosmetic preparations are packaged in a dispenser from which they are sprayed either by mechanical force or with the aid of a blowing agent through a valve. Both methods have obvious drawbacks. While pump sprays are not usually suitable for a lasting, even spray application of cosmetic hair preparations, aerosol sprays are based on the use of blowing agents or propellants, which do not have any cosmetic effect themselves, and which may also present a risk of injury to consumers if misused.

Against this background there is a need for alternative ways to atomize cosmetic hair preparations. Flash evaporation has proven effective as such an alternative spraying method. In this method, which is described for example in International Patent Application WO 2001/83071 A1 (Henkel), a liquid or paste-like solvent-containing composition is heated in an enclosed space to a temperature above the boiling point of the solvent, whereby overpressure is generated in the composition. When the pressure is released (throttling), the liquid evaporates and can then be atomized, by employing a suitable nozzle for example.

But while flash evaporation is generally suitable for the spray application of cosmetic hair preparations, at the same time not all preparations can be atomized by employing a flash evaporation process. This is due on the one hand to the heating of the cosmetic preparation that is needed for flash evaporation, and on the other hand to the specifics of the spray mist produced by flash evaporation, for example the droplet size and droplet density produced in the spray mist.

### BRIEF SUMMARY

A cosmetic product is provided herein. The cosmetic product includes a cosmetic preparation including, relative to the total weight thereof, from about 60 to about 98 wt % of at least one polar solvent and from about 0.5 to about 20 wt % of at least one copolymer. The at least one copolymer is of vinylpyrrolidone, vinyl acetate, and optionally further monomers. The cosmetic product further includes a device for flash evaporation of cosmetic preparation.

A cosmetic composition is also provided herein. The cosmetic composition includes, relative to the total weight thereof, from about 60 to about 98 wt % of at least one polar solvent and from about 0.5 to about 20 wt % of at least one copolymer. The at least one copolymer is of vinylpyrrolidone, vinyl acetate, and optionally further monomers. The cosmetic composition is utilized as a material in a device for flash evaporation.

A method for the temporary reshaping of keratin-containing fibers is also provided herein. The method includes applying a cosmetic composition to the keratin-containing fibers utilizing an apparatus for flash evaporation. The cosmetic composition includes, relative to the total weight thereof, from about 60 to about 98 wt % of at least one polar solvent and from about 0.5 to about 20 wt % of at least one copolymer. The at least one copolymer is of vinylpyrrolidone, vinyl acetate, and optionally further monomers.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present disclosure was therefore to provide specific cosmetic hair preparations for temporary reshaping of keratin-containing fibers which by their chemical and physical properties are suitable for targeted spray application by employing an apparatus for flash evaporation in order to achieve a high level of hold, particularly a high level of long-term hold and a high volumizing effect. It has been found that, of the many polymer preparations known to be effective hair cosmetics, solvent-containing preparations based on a copolymer of vinylpyrrolidone and vinyl acetate lend themselves particularly to achieving this object.A first object of the present disclosure is thus a cosmetic product, comprising

a) a cosmetic preparation comprising, relative to the total weight thereof,
a1) from about 60 to about 98 wt % of at least one polar solvent;
a2) from about 0.5 to about 20 wt % of at least one copolymer of
   i) vinylpyrrolidone
   ii) vinyl acetate
   iii) and optionally further monomers
      b) a device for flash evaporation of cosmetic preparation a). Cosmetic preparation a) is preferably liquid. Cosmetic preparation a) may be present as a solution or dispersion, for example as an emulsion or suspension. Preferred cosmetic preparations a) are in the form of a solution or a suspension.

The novel cosmetic preparation contains, as a first essential component from about 60 to about 98 wt % of at least one polar solvent a1). Preferred cosmetic products are characterized in that the proportion by weight of the polar solvent a1) in the total weight of the cosmetic preparation a) from about 70 to about 98 wt %, preferably from about 80 to about 95% by weight. Corresponding compositions are characterized by a good cosmetic effect together with good applicability.

To improve the performance properties of inventive cosmetic preparations while minimizing the thermal load of any active or auxiliary substances during the flash evaporation process, it has proved advantageous to use polar solvents a1) that have a boiling temperature (20° C., 1013 mbar) between about 50 and about 110° C., preferably from about 70 to about 105° C. In this context, ethanol, isopropanol and water have been found to be particularly advantageous, and for this reason they are preferred for use as polar solvents a1).

Particularly preferred polar solvents a1) or solvent systems are
polar solvents a1) comprising more than about 20 wt %, preferably more than about 30% by weight and particularly more than about 40 wt % of ethanol, relative to the total weight of the polar solvent in each case;
polar solvents a1) comprising, relative to the total weight of the polar solvent in each case, more than about 80 wt %, preferably more than about 90 wt % and particularly more than about 95 wt % of a mixture of ethanol and water, wherein the weight ratio of ethanol to water is preferably in the range from about 10:1 to about 3:1, preferably from about 8:1 to about 4:1;
polar solvents a1) comprising more than about 80 wt %, preferably more than about 88 wt % and particularly more than about 92 wt % water, relative to the total weight of the polar solvent in each case.

Very particularly preferred cosmetic preparations are characterized in that the proportion of volatile constituents is not more than about 55 wt %. The group of these volatile components includes, among others, the polar solvent ethanol. In other words, very particularly preferred cosmetic preparations are therefore characterized by the fact that the proportion by weight of ethanol to the total weight of the cosmetic preparation is not more than 55 wt %, preferably from about 10 to about 55 wt %, particularly preferably from about 25 to about 55 wt % and particularly from about 40 to about 55 wt %.

A further preferred object of the present disclosure is therefore a cosmetic product comprising
a) a cosmetic preparation comprising, relative to the total weight thereof
a1) from about 60 to about 98 wt % of at least one polar solvent, wherein the percentage of ethanol in the total weight of the cosmetic preparation is not more than 55 wt %, preferably from about 10 to about 55 wt %, particularly preferably from about 25 to about 55 wt % and particularly from about 40 to about 55 wt %;
a2) from about 0.1 to about 20 wt % of at least one copolymer of the monomers
i) vinylpyrrolidone
ii) vinyl acetate
iii) and optionally other monomers;
b) a device for flash evaporation of cosmetic preparation a) comprising
b1) a receptacle b1) which is openable and closable by means of a valve
b2) a heating device with which it is possible to heat cosmetic preparation in receptacle b1)
b3) a nozzle b3) with which cosmetic preparation a) can be atomized.

A preferred combination of polar solvents a1) comprises water and ethanol, wherein
the proportion by weight of water and ethanol in the total weight of polar solvent a1) is preferably at least about 60 wt %, more preferably at least about 80 wt %, particularly preferably at least
the proportion by weight of water to ethanol is in the range from about 5:1 to about 1:5, preferably from about 2:1 to about 1:2, particularly from about 5:4 to about 4:5. Another preferred object of the present disclosure is therefore a cosmetic product comprising
a) a cosmetic preparation comprising, relative to the total weight thereof
a1) from about 60 to about 98 wt % of at least one polar solvent, wherein
   the proportion by weight of water and ethanol in the total weight of polar solvent a1) is preferably at least about 60 wt %, more preferably at least about 80 wt %, particularly preferably at least about 90 wt % and especially at least about 95 wt %, and further preferably
   the proportion by weight of water to ethanol is in the range from about 5:1 to about 1:5, preferably from about 2:1 to about 1:2, particularly from about 5:4 to about 4:5.
a2) from about 0.1 to about 20 wt % of at least one copolymer of the monomers
i) vinylpyrrolidone
ii) vinyl acetate
iii) and optionally other monomers;
b) a device for flash evaporation of cosmetic preparation a) comprising
b1) a receptacle b1) which is openable and closable by means of a valve
b2) a heating device with which it is possible to heat cosmetic preparation in receptacle b1)
b3) a nozzle b3) with which cosmetic preparation a) can be atomized.

A second essential component of cosmetic compositions as contemplated herein is the copolymer a2). With regard to manufacturability, ease of application and cosmetic effect of inventive cosmetic compositions, it has proven advantageous if the proportion by weight of copolymer a2) of the total weight of cosmetic preparation a) is from about 1.0 to about 18 wt %, preferably from about 4.0 to about 15 wt %.

Copolymer a2) may be derived from the monomers i) vinylpyrrolidone and ii) vinyl acetate, and optionally other monomers. These vinylpyrrolidone-vinyl acetate copolymers may contain vinyl-pyrrolidone and vinyl acetate in different ratios. Vinylpyrrolidone-vinyl acetate copolymers with a higher vinylpyrrolidone content, with a vinylpyrrolidone content greater than about 60%, are soluble in water. On the other hand, vinylpyrrolidone-vinyl acetate copolymers with a lower vinylpyrrolidone content are soluble in ethanol. It is preferable for the purposes of the disclosure if vinylpyrrolidone-vinyl acetate copolymer a2) has a weight ratio of vinylpyrrolidone to vinyl acetate in the range from about 30:70 to about 70:30.

Preferred copolymers a2) preferably consist of at least about 90 wt %, more preferably at least about 95 wt % and particularly at least about 97 wt % of the monomers i) pyrrolidone, and ii) vinyl acetate. Particularly preferred copolymers e) are obtained using the monomers i) vinylpyrrolidone and ii) vinyl acetate alone.

The copolymers a2) described above are available commercially for example, under the tradenames Luviskol® VA 37, Luviskol® VA 55, Luviskol® VA 64, Luviskol® VA 73 (INCI designation: VP/VA Copolymer, CAS number 25086-89-9), all of which are marketed by BASF SE.

A particularly preferred object of the present patent application is therefore a cosmetic product comprising
a) a cosmetic preparation comprising, relative to the total weight thereof
a1) from about 80 to about 95 wt % of at least one polar solvent;
a2) from about 4.0 to about 15 wt % of at least one copolymer of
i) vinylpyrrolidone
ii) vinyl acetate
iii) and optionally other monomers;
b) a device for flash evaporation of cosmetic preparation a).

Besides cosmetic preparation a), the cosmetic products of the disclosure further include an apparatus for flash evaporation. Within the scope of the present application, the term “flash evaporation” means the generation of vapor by lowering the pressure in an enclosed space that is filled with a liquid and under overpressure (compared with the environment). A corresponding overpressure may be produced for example by heating a quantity of the cosmetic preparation a) to a temperature T₁ in an enclosed space. In the enclosed space, the liquid is has a pressure saturation pressure pi at the given temperature T₁. If the enclosed space is opened, by employing a valve for example, and transformed into a relaxation space which is not under overpressure, where pressure pₒ<p₁, the pressure in the previously enclosed space falls, and as the new pressure level is established through the space the cosmetic preparation a) and the solvent contained in the cosmetic preparation or parts thereof are vaporized. The resulting vapor or spray mist may be used for the application of specific cosmetic preparations.

Thus, if cosmetic preparation a) is heated in an enclosed space beginning from standard conditions (To=25° C., po=1,000 bar), apart from increased temperature this results in increased pressure in cosmetic preparation a). This increased pressure can be relieved in a relaxation space to a pressure po, ambient air pressure for example (po=1, 000 bar), thereby causing at least partial evaporation of cosmetic preparation a).

Cosmetic preparation a) may be relaxed directly in the space in which it was previously heated. Alternatively, the heated and pressurized cosmetic preparation a) may also be transported into a second space after heating, in which the pressure is then relieved.

In other words, flash evaporation is a method in which cosmetic preparation a) is heated in an enclosed container by a heater to temperatures above the ambient temperature, wherein a pressure above ambient pressure is created inside the container and the heated cosmetic preparation a) under increased pressure is then depressurized from the container into the environment.

An apparatus for flash evaporation is therefore a device comprising a container and a heater that is configured such that a cosmetic composition a) may be heated by employing the heater in the closed container to temperatures above the ambient temperature in such manner that a pressure above ambient pressure is created inside the container and the cosmetic preparation a) which is heated and under increased pressure may be released from the container into the surrounding atmosphere.

Simultaneously with or after the pressure relief, cosmetic preparation a) may be forwarded to a nozzle such that it is possible to influence properties of the vapor or spray generated by the flash evaporation, for example, particularly the droplet size or droplet density as well as the spraying range and the shape of the spray cone. The use of nozzles, preferably atomizing nozzles, is therefore preferred. The specific nozzle type or nozzle design is defined specifically on the basis of the respective spray mist characteristics.

In summary, a preferred apparatus for flash evaporation includes
b1) a container b1) which can be opened and closed by means of a valve and which defines the enclosed inner space in which the cosmetic preparation may be accommodated,
b2) a heater b2), which makes it possible to heat a cosmetic preparation inside container b1).

Use of an additional nozzle b3) which enables atomization of the cosmetic preparation a) as it escapes from the container is particularly preferred. Instead of a valve, a similarly acting closing element which can block or release an associated opening in the container by a corresponding change in position, may also be used.

A preferred object of the present disclosure is a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a) wherein the apparatus for flash evaporation comprises a container b1) and a heater b2) and is configured such that
   the cosmetic preparation a) can be accommodated in the interior of container b1),
   the interior space of container b1) which is at least partly filled with cosmetic preparation a) may be closed off,
   cosmetic preparation a) may be heated in the closed interior of container b1) by heater b2), increasing the pressure therein.

A particularly preferred object of the present disclosure is therefore a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of the cosmetic preparation a) wherein the device for flash evaporation comprises a container b1) and a heater b2) and is configured such that
   the cosmetic preparation a) can be accommodated in the interior of container b1),
   the interior space of container b1) which is at least partly filled with cosmetic preparation a) may be closed off,
   cosmetic preparation a) may be heated in the closed interior of container b1) by heater b2), increasing the pressure therein,
   the heated cosmetic preparation a) can be relaxed from the interior of container b1) into the environment, thereby reducing the pressure.

The container b1) in which the cosmetic preparation is heated is designed in a way that makes it possible to close this container off from the environment completely while cosmetic preparation a) is being heated, and after the preparation has been heated to open the container to allow flash evaporation of cosmetic preparation a). This may be ensured for example by a flow controlling component, particularly a valve.

The container b1) in which the cosmetic preparation is heated preferably communicates with another container, from which the quantity of cosmetic preparation intended to undergo flash evaporation prior is transferred to container b1) prior to heating. Access between this reservoir and container b1) is openable and closable by employing an appropriate device, such as a valve. This further container is preferably designed as a reservoir, that is to say it preferably contains several times, for example more than ten times, preferably more than fifty times the quantity of the cosmetic preparation required for an evaporation operation. In other words, the volume of the further container/reservoir is preferably several times, for example more than ten times, preferably more than twenty times and in particular more than fifty times greater than the volume of the container b1).

A further particularly preferred object of the present disclosure is therefore a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a), comprising
b1) a container b1) which may be closed and opened by employing a valve
b2) a heater b2), which makes it possible to heat a cosmetic preparation inside container b1)
b3) a nozzle b3) which enables atomization of the cosmetic preparation a).
c) a reservoir for cosmetic preparation a), from which cosmetic preparation a) may pass into container b1), wherein
   the gateway between the reservoir and container b1) has a flow control component, such that the Flow of cosmetic preparation a) from the reservoir into vessel b1) can be interrupted;
   the volume of the reservoir is at least ten times greater, preferably at least twenty times greater and particularly at least fifty times greater than that of container b1).

The reservoir is not a pressure vessel, and the cosmetic composition in the reservoir is not under pressure, in other words the pressure inside the reservoir is equal to the ambient pressure (also called air pressure or atmospheric pressure). Accordingly, cosmetic products of such kind do not comprise a propellant, for example. The cosmetic product also has no pumping device that is capable of releasing or spraying the cosmetic preparation into the environment without the action of the apparatus for flash evaporation.

A particularly preferred object of the present disclosure is therefore a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a), comprising
b1) a container b1) which may be closed and opened by means of a valve
b2) a heater b2), which makes it possible to heat a cosmetic preparation inside container b1)
b3) a nozzle b3) which enables atomization of the cosmetic preparation a).
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least ten times greater, preferably at least fifty times greater than that of container b1)
   the pressure in the interior of the reservoir corresponds to ambient pressure.

A most particularly preferred object of the present disclosure is therefore a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a), comprising
b1) a container b1) which may be closed and opened by employing a valve
b2) a heater b2), which makes it possible to heat a cosmetic preparation inside container b1)
b3) a nozzle b3) which enables atomization of the cosmetic preparation a).
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least ten times greater, preferably at least fifty times greater than that of container b1);
   the pressure inside the reservoir corresponds to ambient pressure.

A most particularly preferred object of the present disclosure is therefore a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a), comprising
b1) a container b1) which may be closed and opened by employing a valve
b2) a heater b2), which makes it possible to heat a cosmetic preparation inside container b1)
b3) a nozzle b3) which enables atomization of the cosmetic preparation a).
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least ten times greater, preferably at least fifty times greater than that of container b1);
   the pressure inside the reservoir corresponds to ambient pressure and contains no propellant.

In addition, cosmetic products are preferred which comprise
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a), comprising
b1) a container b1) which may be closed and opened by employing a valve
b2) a heater b2), which makes it possible to heat a cosmetic preparation inside container b1)
b3) a nozzle b3) which enables atomization of the cosmetic preparation a).
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least ten times greater, preferably at least fifty times greater than that of container b1);
   the pressure inside the reservoir corresponds to ambient pressure
      wherein the cosmetic product does not have a pumping device which is suitable for releasing the cosmetic preparation a) without the action of the apparatus for flash evaporation or spray. In summary, a particularly preferred object of the present disclosure is therefore a cosmetic product comprising
a) a cosmetic preparation comprising, relative to the total weight thereof
a1) from about 60 to about 98 wt % of at least one polar solvent;
a2) from about 0.05 to about 20 wt % of at least one copolymer of
i) vinylpyrrolidone
ii) vinyl acetate
iii) and optionally other monomers;
b) a device for flash evaporation of cosmetic preparation a), comprising
b1) a container b1) which may be closed and opened by employing a valve
b2) a heater b2), which makes it possible to heat a cosmetic preparation inside container b1)
b3) a nozzle b3) which enables atomization of the cosmetic preparation a).
c) a reservoir for cosmetic preparation a), from which cosmetic preparation a) may pass into container b1), wherein
   the gateway between the reservoir and container b1) has a flow control component, such that the flow of cosmetic preparation a) from the reservoir into vessel b1) can be interrupted;
   the volume of the reservoir is at least ten times greater, preferably at least fifty times greater than that of container b1);
   the pressure inside the reservoir corresponds to ambient pressure
      wherein the cosmetic product does not have a pumping device which is suitable for releasing the cosmetic preparation a) without the action of the apparatus for flash evaporation or spray. Besides the two above-described components a1) and a2) the cosmetic preparations as contemplated herein a) may contain other active or auxiliary substances, wherein those active or auxiliary substances are particularly preferred which improve the manufacturability, ease of application and/or cosmetic effect of inventive cosmetic preparations.

A first example of preferred active and auxiliary agents are cationic surfactants a3). Preferred cationic surfactants a3) are selected from quaternary ammonium compounds, esterquats and amidoamines. The cationic surfactants are contained in cosmetic preparation a) in quantities relative to the total weight thereof from about 0.01 to about 4.0 wt %, preferably from about 0.05 to about 2.0 wt % and particularly from about 0.1 to about 1.0 wt %. Cationic surfactants a3) from the group of quaternary ammonium compounds are particularly preferred.

Preferred quaternary ammonium compounds are ammonium halides, in particular chlorides and bromides, such as alkyltrimethylammonium chlorides, dialkyldimethylammonium and trialkylmethylammonium chlorides, e.g. cetyltrimethylammonium chloride, stearyltrimethylammonium chloride, distearyldimethylammonium chloride, lauryldimethylammomonium chloride, lauryldimethylbenzylammomonium chloride and tricetylmethylammonium chloride, as well as imidazolium compounds known by the INCI names Quaternium-27 and Quaternium-83. The long alkyl chains of the abovementioned surfactants preferably have 10 to 18 carbon atoms. Very particularly preferred cosmetic preparations as contemplated herein a) are characterized in that they contain from about 0.01 to about 4.0 wt %, preferably from about 0.05 to about 2.0 wt % and in particular from about 0.1 to about 1.0 wt % (C12 to C18)-alkyltrimethylammonium salt(s) relative to the total weight thereof.

Another example of preferred active and auxiliary agents are film-forming polymers a4), the use of which is preferred in cosmetic products as contemplated herein.

Suitable film-forming polymers a4) are permanently and temporarily cationic, anionic, nonionic or amphoteric polymers. These film-forming polymers may be synthetic or of natural origin. Preferred cosmetic preparations a) contain from about 0.1 to about 20 wt %, preferably from about 0.5 to about 15 wt %, and particularly from about 1.0 to about 10 wt % of a film-forming polymer a4) relative to the total weight thereof. This polymer a4) is different from polymer a2).

Examples of usual film-forming polymers a4) are Acrylamide/Ammonium Acrylate copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates copolymer, Acrylates/Acetoacetoxyethyl Methacrylate Copolymer, Acrylates/Acrylamide Copolymer, Acrylates/Ammonium Methacrylate copolymer, Acrylates/t-Butylacrylamide copolymer, Acrylates copolymer, Acrylates/C1-2 Succinates/Hydroxyacrylates copolymer, Acrylates/Lauryl Acrylate/Stearyl Acrylate/Ethylamine Oxide Methacrylate copolymer, Acrylates/Octylacrylamide copolymer, Acrylates/Octylacrylamide/Diphenyl Amodimethicone copolymer, Acrylate/Stearyl Acrylate/Ethylamine Oxide Methacrylate copolymer, Acrylates/VA copolymer, Acrylates/VP copolymer, Adipic Acid/Diethylenetri amine copolymer, Adipic Acid/Dimethylaminohydroxypropyl Diethylenetriamine copolymer, Adipic Acid/Epoxypropyl Diethylenetriamine copolymer, Adipic Acid/Isophthalic Acid/Neopentyl Glycol/Trimethylolpropane copolymer, Allyl Stearate/VA copolymer, Aminoethylacrylate Phosphate/Acrylates copolymer, Aminoethylpropanediol-Acrylates/Acrylamide copolymer, Aminoethylpropanediol-AMPD-Acrylates/Diacetoneacrylamide copolymer, Ammonium VA/Acrylates copolymer, AMPD-Acrylates/Diacetoneacrylamide copolymer, AMP-Acrylates/Allyl Methacrylate copolymer, AMP Acrylates/C1-18 Alkyl Acrylates/C1-8 Alkyl Acrylamide copolymer, AMP Acrylates/Diacetoneacrylamide copolymer, AMP-Acrylates/Dimethylaminoethylmethacrylate copolymer, Bacillus/Rice Bran Extract/Soybean Extract Ferment Filtrates, Bis-Butyloxyamodimethicone/PEG-60 copolymer, Butyl Acrylate/Ethylhexyl Methacrylate copolymer, Butyl Acrylate/Hydroxypropyl Dimethicone Acrylate copolymer, Butylated PVP, Butyl Ester of Ethylene/MA copolymer, Butyl Ester of PVM/MA copolymer, Calcium/Sodium PVM/MA copolymer, Corn Starch/Acrylamide/Sodium Acrylate copolymer, Diethylene Glycolamine/Epichlorohydrin/Piperazine copolymer, Dimethicone Crosspolymer, Diphenyl Amodimethicone, Ethyl Ester of PVM/MA copolymer, Hydrolyzed Wheat Protein/PVP Crosspolymer, Isobutylene/Ethylmaleimide/Hydroxyethylmaleimide copolymer, Isobutylene/MA copolymer Isobutylmethacrylate/Bis-Hydroxypropyl Dimethicone Acrylate copolymer, Isopropyl Ester of PVM/MA copolymer, Lauryl Acrylate Crosspolymer, Lauryl Methacrylate/Glycol Dimethacrylate Crosspolymer, MEA-sulfites, Methacrylic Acid/Sodium Acrylamidomethyl Propane Sulfonate copolymer, Methacryloyl Ethyl Betaine/Acrylates copolymer, Octylacrylamide/Acrylates/Butylaminoethyl Methacrylate copolymer, PEG/PPG-25/25 Dimethicone/Acrylates copolymer, PEG-8/SMDI copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquatemium-1, Polyquatemium-2, Polyquatemium-4, Polyquatemium-5, Polyquaternium-6, Polyquatemium-7, Polyquatemium-8, Polyquatemium-9, Polyquatemium-10, Polyquatemium-11, Polyquatemium-12 , Polyquatemium-13, Polyquatemium-14, Polyquatemium-15, Polyquatemium-16, Polyquatemium-17, Polyquatemium-18, Polyquatemium-19, Polyquatemium-20, Polyquatemium-22, Polyquatemium-24, Polyquatemium-27, Polyquatemium-28, Polyquatemium -29, Polyquatemium-30, Polyquatemium-31, Polyquatemium-32, Polyquatemium-33, Polyquatemium-34, Polyquatemium-35, Polyquatemium-36, Polyquatemium-39, Polyquatemium-45, Polyquatemium-46, Polyquatemium-47, Polyquatemium-48 , Polyquatemium-49, Polyquatemium-50, Polyquatemium-55, Polyquatemium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA copolymer, Potassium Ethyl Ester of PVM/MA copolymer, PPG-70 Polyglyceryl-10 Ether, PPG-12/SMDI copolymer, PPG-51/SMDI copolymer, PPG-10 Sorbitol, PVM/MA copolymer, PVP, PVPNA/Itaconic Acid copolymer, PVP/VA/Vinyl Propionate copolymer, Rhizobian Gum, Rosin Acrylate, Shellac, Sodium Butyl Ester of PVM/MA copolymer, Sodium Ethyl Ester of PVM/MA copolymer, Sodium Polyacrylate, Sterculia Urens Gum, Terephthalic Acid/Isophthalic Acid/Sodium Isophthalic Acid Sulfonate/Glycol copolymer, Trimethylolpropane Triacrylate Trimethylsiloxysilyl-carbamoyl Pullulan, VA/Crotonates copolymer, VA/Crotonates/Methacryloxybenzophenone-1 copolymer, VA/Crotonates/Vinyl Neodecanoate copolymer, VA/Crotonates/Vinyl Propionate copolymer, VA/DBM copolymer, VA/Vinyl Butyl Benzoate/Crotonates copolymer, Vinylamine/Vinyl Alcohol copolymer, Vinyl Caprolactam/VP/Dimethylaminoethyl Methacrylate copolymer, VP/Acrylates/Lauryl Methacrylate copolymer, VP/Dimethylaminoethylmethacrylate copolymer, VP/DMAPA Acrylates copolymer, VP/Hexadecene copolymer, VP/VA copolymer, VP/Vinyl Caprolactam/DMAPA Acrylates copolymer, Yeast Palmitate and Styrene/VP copolymer.

Particularly preferred are cosmetic products, characterized in that the film-forming polymer a4) is selected from the group of anionic polymers, preferably from the group of copolymers of acrylic acid and methacrylic acid.

A particularly preferred anionic acrylate copolymer a4) consists of at least the following monomer units: at least one (meth)acrylic acid unit (1), at least one (meth)acrylic acid alkylester unit (2) and at least one (meth)acrylic acid hydroxyalkyl ester unit (3). As contemplated herein, this preferred copolymer a4) may be constructed from other monomer units. According to variants of the disclosure, however, copolymer a4) is constructed only from units (1), (2) and (3), i.e. it consists of units derived from these monomer units.

The at least one methacrylic acid unit (1) may be a methacrylic or acrylic acid unit.

The alkyl radical of the (meth)acrylic acid alkyl ester unit (2) is preferably a C1-C8 alkyl radical, which may be linear or branched. Examples of alkyl radicals are methyl, ethyl, propyl, isopropyl, 1-butyl, 2-butyl, iso-butyl, tert-butyl, linear or branched pentyl, linear or branched hexyl, linear or branched heptyl, and linear or branched octyl. More preferably, the alkyl group is a C1 to C5 alkyl group. According to one variant of the disclosure, two or more (meth)acrylic acid alkyl ester units (2) are included, which differ in the carbon number of the alkyl group. For example, a C1-C3-alkyl methacrylate and a C2-C5 alkyl acrylate unit C2-C5 are included.

The hydroxyalkyl radical of the (meth)acrylic acid hydroxyalkyl ester unit (3) may be a hydroxy-C1-C10-alkyl radical, preferably a hydroxy-C2-C5 alkyl radical. In a preferred embodiment, the (meth)acrylic acid hydroxyalkyl ester unit (3) is (meth)acrylic acid hydroxyethyl ester.

The proportional content of units (1), (2) and (3) in the acrylate resin a4) may vary within wide limits. The proportion of unit (1) in the acrylate copolymer is preferably from about 2 to about 50 wt %, more preferably from about 5 to about 30 wt %. The proportion of unit (2) in the acrylate copolymer is preferably from about 5 to about 95 wt %, more preferably from about 45 to about 90 wt %. The proportion of unit (3) in the acrylate copolymer is preferably from about 2 to about 70 wt %, more preferably from about 5 to about 30 wt %.

Suitable anionic acrylate a4) are commercially available with INCI name acrylates / hydroxy ester acrylates copolymer. Most preferable is the anionic acrylate copolymer (a) Acudyne® 1000 from The Dow Chemical Company.

Another preferred anionic acrylate copolymer a4) comprises structural units with formula (4) wherein R¹ represents a methyl group and R2 represents a methyl group, and structural units of formula (4) wherein R¹ represents a hydrogen atom and R2 represents a butyl group (particularly an n-butyl group), and structural units of formula (5) wherein R3 represents a methyl group and R4 represents a 2-hydroxyethyl group and structural units of formula (6) wherein R7 represents a methyl group at least one structural unit of formula (7) wherein R5 and R6 are independently a hydrogen atom or a (C1-C6) alkyl group, preferably hydrogen

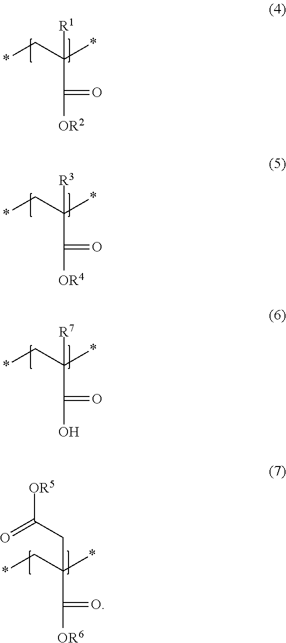

A particularly preferred polymer has the INCI nomenclature Acrylates/C1-2 succinates/Hydroxyacrylates copolymer. It can be purchased for example from Dow under the trade name Acudyne LT-120 (INCI nomenclature: Acrylates/C1-2 succinates/Hydroxyacrylates copolymer).

A second group of particularly preferred film-forming polymers a4) comprises amphoteric setting polymer C based on
at least one monomer C1 selected from acrylic acid, methacrylic acid, acrylic acid alkyl esters and methacrylic acid alkyl esters, and
at least one amphoteric monomer C2 of formula C2

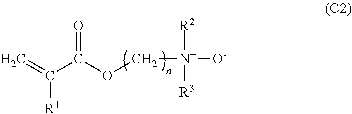

wherein
R¹ represents H or CH3,
R2 and R3 each independently represent optionally branched C₁₋₁₀ alkyl and
n is an integer from 1 to 20.

For the purposes of the present variant of the disclosure, further amphoteric fixing polymers which are formed from the monomers mentioned are understood to include only those copolymers C which, besides polymer units resulting from the incorporation of said monomers C1 and C2 in the copolymer, contain at most 5 wt %, preferably not more than 1 wt % of polymer units that trace back to the incorporation of other monomers. Copolymers C are preferably composed exclusively of polymer units resulting from the incorporation of said monomers C1 and C2 in the copolymer.

Suitable monomers C1 are acrylic acid, methacrylic acid, acrylic acid-C₁₋₂₀-alkyl esters and methacrylic acid C₁₋₂₀ alkyl esters. Particularly suitable is a monomer C1 selected from acrylic acid, methacrylic acid, methyl acrylate, methyl methacrylate, ethyl acrylate, ethyl methacrylate, propyl acrylate, propyl methacrylate, isopropyl acrylate, isopropyl methacrylate, lauryl acrylate, lauryl methacrylate, cetyl acrylate, cetyl methacrylate, stearyl acrylate and stearyl methacrylate, most preferably from acrylic acid, methacrylic acid, methyl acrylate, methyl methacrylate, ethyl acrylate, ethyl methacrylate, lauryl acrylate, lauryl methacrylate, stearyl acrylate and stearyl methacrylate.
Suitable monomers C2 are (meth)acryloylalkylaminoxides of formula C2 wherein R2 and R3 each independently represent methyl, ethyl, propyl, isopropyl, butyl, isobutyl or tert-butyl, particularly preferably methyl. Suitable monomers C2 are further selected from at least one monomer from the group formed from (meth)acryloyl alkyl amine oxides of formula C2, wherein n is an integer from 1 to 5, preferably an integer of 1 to 3 and particularly preferably 2.

Monomers C2 are preferably also selected from at least one monomer from the group formed from (meth)acryloyl alkyl amine oxides of formula C2 wherein R¹ stands for CH3. Particularly suitable are monomers C2 selected from at least one monomer from the group which is formed from (meth)acryloyl alkyl amine oxides of formula C2 wherein R2 and R3 independently represent methyl, ethyl, propyl, isopropyl, butyl, isobutyl or tert. butyl, particularly preferably methyl, n stands in each case for an integer from 1 to 5, preferably an integer from 1 to 3 and particularly preferably 2, and R1 is CH3 in each case. Monomer C2 is very particularly suitably selected from at least one monomer from the group formed from (meth)acryloyl alkyl amine oxides of formula C2, wherein R, R2 and R3 are CH3 and n is 2. Preferred cosmetic products are characterized in that the film-forming polymer a4) is selected from the group of copolymers of methacryloylethyl-N, N-dimethylaminoxide.

In a particularly suitable embodiment, the cosmetic preparation as contemplated herein contains at least one amphoteric setting polymer formed from
least two C1 monomers wherein the first monomer is selected from acrylic acid, methacrylic acid, methyl acrylate, methyl methacrylate, ethyl acrylate, ethyl methacrylate, propyl acrylate, propyl methacrylate, isopropyl acrylate and isopropyl methacrylate, and the second monomer is selected from stearyl acrylate and stearyl methacrylate, and
monomer C2 is selected from methacryloyl ethyl amine oxide, in particular methacryloyl ethyl-N, N-dimethylamine (in the formula (A2): R1=CH 3, n=2, R2 and R3=CH).

These copolymers are also known and commercially available, for example with trade names Diaformer Z-611, Diaformer Z-612, Diaformer Z-613, Diaformer Z-631, Diaformer Z-633, Diaformer Z-651, Diaformer Z-711N, Diaformer Z-712N and Diaformer Z-731N from Clariant, wherein the use of Diaformer Z-712N and Diaformer Z-651 is particularly preferred.

A third group of particularly preferred film-forming polymers a4) comprises copolymers a4) which can be traced back to the monomers i) N-tert-octylacrylamide, ii) acrylic acid, iii) tert-butylaminoethyl methacrylate and optionally other monomers.
Preferred copolymers a4) preferably consist of at least about 90 wt %, more preferably at least about 95 wt % and in particular at least about 97 wt % of the monomers N-tert-octyl acrylamide, acrylic acid and tert.-butylaminoethyl methacrylate. Particularly preferred copolymers a4) were obtained exclusively from the monomers N-tert-octyl acrylamide, acrylic acid and tert.-butylaminoethyl methacrylate.

Particularly preferred are copolymers a4) from the monomers i) N-tert-octylacrylamide, ii) acrylic acid, iii) tert-butylaminoethyl methacrylate, iv) methyl methacrylate and v) hydroxypropyl methacrylate.

The copolymers a3) described above are marketed for example with the name Amphomer® (INCI name: octylacrylamide/acrylates/butylaminoethyl methacrylate copolymer; CAS number 70801-07-9) by National Starch.

Other suitable active or auxiliary substances include in particular care products.

As a care substance, the agent may contain for example at least one protein hydrolysate and/or one of its derivatives. Protein hydrolysates are product mixtures obtained by acid-, base- or enzyme-catalyzed degradation of proteins (albumins). The term protein hydrolysate as contemplated herein is also understood to include total hydrolysates as well as individual amino acids and their derivatives and mixtures of different amino acids. The molecular weight of the protein hydrolysated usable as contemplated herein is between about 75, the molecular weight of glycine, and about 200,000, the molecular weight is preferably about 75 to about 50,000 and very particularly preferably about 75 to about 20,000 Daltons.

The composition of the disclosure may further comprise at least one vitamin, one provitamin, one vitamin precursor and/or one of the derivatives thereof as a care substance. In this case, those vitamins, provitamins and vitamin precursors are preferred as contemplated herein that are commonly assigned to the groups A, B, C, E, F and H.

Further care substances are panthenol, caffeine, nicotinamide and sorbitol. The agents as contemplated herein may further comprise at least one plant extract, but also mono- or oligosaccharides and/or lipids as the care substance.

The compositions of some cosmetic preparations that are particularly preferred as contemplated herein are listed in the following tables (values in wt % relative to the total weight of the cosmetic composition unless otherwise specified). With respect to further preferred variants of these particularly preferred compositions, the earlier notes on the inventive cosmetic preparations a) still apply mutatis mutandis.

| | Formula 1 | Formula 2 | Formula 3 | Formula 4 | Formula 5 |
|---|---|---|---|---|---|
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| Optional additives | add 100 | add 100 | add 100 | add 100 | add 100 |
| | Formula 6 | Formula 7 | Formula 8 | Formula 9 | Formula 10 |
| Polar solvent a1) | 60 to 98 | 70 to 98 | 80 to 95 | 85 | 91 |
| Copolymer a2)¹⁾ | 0.5 to 20 | 1.0 to 18 | 4.0 to 15 | 12.0 | 7.0 |
| Optional additives | add 100 | add 100 | add 100 | add 100 | add 100 |
| | Formula 11 | Formula 12 | Formula 13 | Formula 14 | Formula 15 |
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| Cationic surfactant | 0.01 to 4.0 | 0.05 to 2.0 | 0.1 to 1.0 | 0.3 | 0.2 |
| a3) | | | | | |
| Optional additives | add 100 | add 100 | add 100 | add 100 | add 100 |
| | Formula 16 | Formula 17 | Formula 18 | Formula 19 | Formula 20 |
| Polar solvent a1) | 60 to 98 | 70 to 98 | 80 to 95 | 85 | 91 |
| Copolymer a2)¹⁾ | 0.5 to 20 | 1.0 to 18 | 4.0 to 15 | 12.0 | 7.0 |
| Cationic surfactant | 0.01 to 4.0 | 0.05 to 2.0 | 0.1 to 1.0 | 0.3 | 0.2 |
| a3) | | | | | |
| Optional additives | add 100 | add 100 | add 100 | add 100 | add 100 |
| | Formula 21 | Formula 22 | Formula 23 | Formula 24 | Formula 25 |
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| (C₁₂-C₁₈)alkyl- | 0.01 to 4.0 | 0.05 to 2.0 | 0.1 to 1.0 | 0.3 | 0.2 |
| trimethyl | | | | | |
| ammonium salt | | | | | |
| Optional additives | add 100 | add 100 | add 100 | add 100 | add 100 |
| | Formula 26 | Formula 27 | Formula 28 | Formula 29 | Formula 30 |
| Polar solvent a1) | 60 to 98 | 70 to 98 | 80 to 95 | 85 | 91 |
| Copolymer a2)¹⁾ | 0.5 to 20 | 1.0 to 18 | 4.0 to 15 | 12.0 | 7.0 |
| (C₁₂-C₁₈)alkyl- | 0.01 to 4.0 | 0.05 to 2.0 | 0.1 to 1.0 | 0.3 | 0.2 |
| trimethyl | | | | | |
| ammonium salt | | | | | |
| Optional additives | add 100 | add 100 | add 100 | add 100 | add 100 |
| | Formula 31 | Formula 32 | Formula 33 | Formula 34 | Formula 35 |
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| Polymer a4) | 0.1 to 20 | 0.5 to 15 | 1.0 to 10 | 1.9 | 0.9 |
| Optional additives | add 100 | add 100 | add 100 | add 100 | add 100 |
| | Formula 36 | Formula 37 | Formula 38 | Formula 39 | Formula 40 |
| Polar solvent a1) | 60 to 98 | 70 to 98 | 80 to 95 | 85 | 91 |
| Copolymer a2)¹⁾ | 0.5 to 20 | 1.0 to 18 | 4.0 to 15 | 12.0 | 7.0 |
| Polymer a4) | 0.1 to 20 | 0.5 to 15 | 1.0 to 10 | 1.9 | 0.9 |
| Optional additives | add 100 | add 100 | add 100 | add 100 | add 100 |
| | Formula 41 | Formula 42 | Formula 43 | Formula 44 | Formula 45 |
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| (Meth)acrylate | 0.1 to 20 | 0.5 to 15 | 1.0 to 10 | 1.9 | 0.9 |
| copolymer | | | | | |
| Optional additives | add 100 | add 100 | add 100 | add 100 | add 100 |
| | Formula 46 | Formula 47 | Formula 48 | Formula 49 | Formula 50 |
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| Methacryloyl- | 0.1 to 20 | 0.5 to 15 | 1.0 to 10 | 1.9 | 0.9 |
| ethyl-N,N- | | | | | |
| dimethyl amine | | | | | |
| oxide | | | | | |
| Optional | add 100 | add 100 | add 100 | add 100 | add 100 |
| additives | | | | | |
| | Formula 51 | Formula 52 | Formula 53 | Formula 54 | Formula 55 |
| Polar solvent a1) | 60 to 98 | 70 to 98 | 80 to 95 | 85 | 91 |
| Copolymer a2) | 0.5 to 20 | 1.0 to 18 | 4.0 to 15 | 12.0 | 7.0 |
| N-tert-octyl | 0.1 to 20 | 0.5 to 15 | 1.0 to 10 | 1.9 | 0.9 |
| acrylamide/acrylic | | | | | |
| acid/tert./butyl- | | | | | |
| aminoethyl | | | | | |
| methacrylate | | | | | |
| copolymer | | | | | |
| Optional additives | add 100 | add 100 | add 100 | add 100 | add 100 |

¹⁾Copolymer with INCI designator VP/VA copolymer

Very particularly preferred cosmetic preparations contain only small amounts of further active substances and auxiliaries besides the above-described components a1) to a4). Cosmetic preparations which are characterized in that the proportion by weight of components a1), a2), and a3) and/or a4) if included, is at least about 80 wt %, preferably at least about 90 wt %, particularly preferably at least about 93 wt % and especially at least about 97 wt % relative to the total weight of the cosmetic preparation, are particularly preferred because of their simple manufacturability and good cosmetic effect. Very particularly preferred cosmetic preparations consist of at least about 80 wt %, preferably at least about 90 wt % and particularly at least about 97 wt % of components a1), a2) and a3) relative to the total weight of the cosmetic preparation.

As explained in the introduction, the cosmetic preparations a) as contemplated herein are particularly suitable for application by employing an apparatus for flash evaporation. A further object of the present application, is therefore the use of a cosmetic formulation a) comprising, relative to the total weight thereof,
a1) from about 60 to about 98 wt % of at least one polar solvent;
a2) from about 0.5 to about 20 wt % of at least one copolymer of
i) vinylpyrrolidone
ii) vinyl acetate
iii) and optionally further monomers as a process material in a device for flash evaporation.
   An object of the present application is also the use of a product as contemplated herein for applying a cosmetic preparation a) to keratin-containing fibers, in particular human hair, or for the temporarily reshaping keratin-containing fibers, particularly human hair.

A method for the temporary reshaping of keratin-containing fibers, particularly human hair, in which the keratin-containing fibers are exposed by employing an apparatus for flash evaporation to a cosmetic preparation a) comprising, relative to the total weight thereof,
a1) from about 60 to about 98 wt % of at least one polar solvent;
a2) from about 0.5 to about 20 wt % of at least one copolymer of
i) vinylpyrrolidone
ii) vinyl acetate
iii) and optionally further monomers
   is a further object of the present application. The cosmetic preparation a) is preferably converted into a spray mist by employing the apparatus for flash evaporation and is then applied to the keratin-containing fibers.

In order to achieve a sufficient spray action, the cosmetic preparation a) is preferably heated to temperatures above the boiling point of the polar solvent or solvent mixture contained in the cosmetic preparation a).

If the polar solvent is water or solvent mixtures with a water content greater than about 50 wt % (relative to the total weight of the solvent mixture), the cosmetic preparation is preferably heated to temperatures above about 100° C., preferably to temperatures between about 100° C. and about 240° C., more preferably to temperatures between about 140° C. and about 160° C.

In the cases in which the polar solvent is water or solvent mixtures with a water content above about 50 wt % (relative to the total weight of the solvent mixture), the overpressure created by heating cosmetic preparation a) is, preferably between about 1.1 and about 8 bar, preferably between about 1.2 and about 4 bar.

A preferred object of the application is a method for changing the colour of keratin-containing fibers, particularly human hair, in which the keratin-containing fibers are exposed to a cosmetic formulation a) comprising, relative to the total weight thereof,
a1) from about 60 to about 98 wt % of at least one polar solvent;
a2) from about 0.5 to about 20 wt % of at least one copolymer of
i) vinylpyrrolidone
ii) vinyl acetate
iii) and optionally further monomers by employing an apparatus for flash evaporation, wherein
   a partial quantity of the cosmetic preparation a) present in a reservoir, inside which there is a pressure corresponding to ambient pressure, is transferred from said reservoir to a container b1)
   then, the communication between the reservoir and container b1) is stopped by a flow control component, such that the flow of cosmetic preparation a) from the reservoir into vessel b1) can be interrupted;
   then, the cosmetic preparation a) in container b1), which is closed off from the environment, is heated by a heater, so that the pressure inside vessel b1) rises to values above ambient pressure, preferably to values between about 1.1 and about 8 bar, particularly to values between about 1.2 and about 4 bar;
   then, the container b1) in which a pressure above ambient pressure prevails opens in such manner as to allow a partial quantity, preferably at least about 50 wt %, more preferably at least about 80 wt % and particularly at least about 90 wt % of the cosmetic preparation in container b1) to escape from container b1) into the environment relieving the pressure that prevailed in container b1) at the time the container was opened, and thus relaxing.

The relaxation of cosmetic preparation a) in the ambient atmosphere is preferably accompanied by the formation of a spray mist of cosmetic preparation a).
The cosmetic preparation a) which has relaxed out of container b1) is preferably applied to keratin-containing fibers, particularly human hair.

Methods in the course of which the cosmetic preparation that is relaxed out of container b1) passes through a nozzle before it is applied to the keratin-containing fibers are particularly preferred.

With respect to further preferred embodiments of the uses as contemplated herein and of the method as contemplated herein, the earlier notes on the inventive cosmetic preparations a) and the device for flash evaporation also apply mutatis mutandis.

The inventive compositions, uses and methods and some preferred embodiments thereof are summarized in the following points:
1. Cosmetic product comprising
a) a cosmetic preparation comprising, relative to the total weight thereof,
a1) from about 60 to about 98 wt % of at least one polar solvent;
a2) from about 0.5 to about 20 wt % of at least one copolymer of
i) vinylpyrrolidone
ii) vinyl acetate
iii) and optionally further monomers
b) a device for flash evaporation of cosmetic preparation a).
2. Cosmetic product according to item 1, characterized in that the device for flash evaporation includes a container b1) and a heater b2) and is designed such that
   the cosmetic preparation a) can be accommodated in the interior of container b1),
   the interior of container b1) which is at least partly filled with cosmetic preparation a) can be closed,
   the cosmetic preparation a) can be heated in the closed interior of container b1) by heater b2), causing an increase in pressure,
   the heated cosmetic preparation a) can be relaxed out of the interior of container b1) into the environment, thereby reducing the pressure.
3. Cosmetic product according to points, characterized in that the proportion by weight of the polar solvent a1) is equal to from about 70 to about 98 wt %, preferably from about 80 to about 95 wt % of the total weight of cosmetic preparation a).
4. Cosmetic product according to points, characterized in that the polar solvent a1) has a boiling temperature (20° C., 1013 mbar) between about 50 and about 110° C., preferably between about 70 and about 105° C.
5. Cosmetic products according to points, characterized in that the polar solvent a1) is selected from the group of ethanol, isopropanol and water.
6. Cosmetic products according to points, characterized in that the proportion by weight of water and ethanol in the total weight of the polar solvent a1) is preferably at least about 60 wt %, more preferably at least about 80 wt %, particularly preferably at least about 90 weight % and most particularly preferably at least about 95 wt %.
7. Cosmetic products according to points, characterized in that the proportion by weight of water in the total weight of the polar solvent a1) is more than about 80 wt %, preferably more than about 88 wt % and particularly more than about 92 wt %.
8. Cosmetic products according to points, characterized in that the proportion by weight of ethanol in the total weight of the cosmetic preparation is not more than about 55 wt %, preferably from about 10 to about 55 wt %, particularly preferably from about 25 to about 55 wt % and especially from about 40 to is 55 wt %.
9. Cosmetic products according to points, characterized in that the polar solvent a1) comprises water and ethanol, and the ratio by weight of water to ethanol is in the range from about 5:1 to about 1:5, preferably in the range from about 2:1 to about 1:2 and particularly in the range from about 5:4 to about 4:5.
10. Cosmetic product according to points, characterized in that the proportion by weight of copolymer a2) is of the total weight of the cosmetic preparation a) is equal to about 1.0 to about 18 wt %, preferably from about 4.0 to about 15 wt %.
11. Cosmetic product according to points, characterized in that cosmetic preparation a) contains, relative to the total weight thereof, from about 0.01 to about 4.0 wt %, preferably from about 0.05 to about 2.0 wt % and particularly from about 0.1 to about 1.0 wt % of a cationic surfactant a3) selected from the groups of quaternary ammonium compounds, esterquats and amidoamines, more preferably from the group of quaternary ammonium compounds, particularly from the group of (C12 to C18)-alkyltrimethylammonium salts.
12. Cosmetic product according to points, characterized in that cosmetic preparation a) contains, relative to the total weight thereof, from about 0.1 to about 20 wt %, preferably from about 0.5 to about 15 wt %, and particularly from about 1.0 to about 10 wt % of a film-forming polymer a4).
13. Cosmetic product according to item 12, characterized in that the film-forming polymer a4) is selected from the group of anionic polymers, preferably from the group of copolymers of acrylic acid and methacrylic acid.
14. Cosmetic product according to item 12, characterized in that the film-forming polymer a4) is selected from the group of copolymers of methacryloylethyl-N, N-dimethylamine oxide.
15. Cosmetic product according to item 12, characterized in that the film-forming polymer a3) is selected from the group of copolymers of i) N-tert-octylacrylamide, ii) acrylic acid, iii) tert-butylaminoethyl methacrylate iv) and optionally further monomers.
16. Cosmetic product according to points, characterized in that cosmetic preparation a) consists of at least about 80 wt %, preferably at least about 90 wt % and particularly at least about 97 wt % of the components a1) and a2) relative to its total weight.
17. Cosmetic product according to points, characterized in that cosmetic preparation a) consists of at least about 80 wt %, preferably at least about 90 wt %, particularly at least about 97 wt % of the components a1), a2) and a3) relative to its total weight.
18. Use of a cosmetic composition a) comprising, relative to the total weight thereof,
a1) from about 60 to about 98 wt % of at least one polar solvent;
a2) from about 0.5 to about 20 wt % of at least one copolymer of
i) vinylpyrrolidone
ii) vinyl acetate
iii) and optionally further monomers as a process material in a device for flash evaporation.
19. Use of a product according to any one of items 1 to 17 for acting on keratin-containing fibers, particularly human hair, with a cosmetic preparation a).
20. Use of a product according to any one of items 1 to 17 for the temporary reshaping of keratin-containing fibers, particularly human hair.
21. Method for the temporary reshaping of keratin-containing fibers, particularly human hair, in which a cosmetic composition a) comprising, relative to the total weight thereof,
a1) from about 60 to about 98 wt % of at least one polar solvent;
a2) from about 0.5 to about 20 wt % of at least one copolymer of
i) vinylpyrrolidone
ii) vinyl acetate
iii) and optionally further monomers is applied to the keratin-containing fibers by employing an apparatus for flash evaporation.
22. The method of item 21, characterized in that
   a partial quantity of the cosmetic preparation a) present in a reservoir, inside which there is a pressure corresponding to ambient pressure, is transferred from said reservoir to a container b1);
   then, the communication between the reservoir and container b1) is stopped by a flow control component, such that the flow of cosmetic preparation a) from the reservoir into vessel b1) can be interrupted;
   then, the cosmetic preparation a) in container b1), which is closed off from the environment, is heated by a heater, so that the pressure inside vessel b1) rises to values above ambient pressure, preferably to values between about 1.1 and about 8 bar, particularly to values between about 1.2 and about 4 bar;
   then, the container b1) in which a pressure above ambient pressure prevails opens in such manner as to allow a partial quantity, preferably at least about 50 wt %, more preferably at least about 80 wt % and particularly at least about 90 wt % of the cosmetic preparation in container b1) to escape from container b1) into the environment relieving the pressure that prevailed in container b1) at the time the container was opened, and thus relaxing.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic product comprising:
a) a cosmetic preparation comprising, relative to the total weight thereof;
a1) from about 60 to about 98 wt % of at least one polar solvent; and
a2) from about 0.5 to about 20 wt % of at least one copolymer of;
i) vinylpyrrolidone,
ii) vinyl acetate, and
iii) and optionally further monomers; and
b) a device for flash evaporation of cosmetic preparation a).

2. Cosmetic product according to claim 1, wherein the device for flash evaporation includes comprises a container b1) and a heater b2) and is designed such that:
the cosmetic preparation a) can be accommodated in the interior of container b1),
the interior of container b1) which is at least partly filled with cosmetic preparation a) can be closed,
the cosmetic preparation a) can be heated in the closed interior of container b1) by heater b2), causing an increase in pressure, and
the heated cosmetic preparation a) can be relaxed out of the interior of container b1) into the environment, thereby reducing the pressure.

3. Cosmetic product according to claim 1, wherein the proportion by weight of the polar solvent a1) is equal to about 70 to about 98 wt % of the total weight of cosmetic preparation a).

4. Cosmetic product according to claim 1, wherein the polar solvent a1) has a boiling temperature (20° C., 1013 mbar) between about 50 and about 110° C.

5. Cosmetic products according to claim 1, wherein the polar solvent a1) is selected from the group of ethanol, isopropanol, water, and combinations thereof.

6. Cosmetic products according to claim 1, wherein the proportion by weight of copolymer a2) of the total weight of cosmetic preparation a) is equal to about 1.0 to about 18 wt %.

7. Cosmetic product according to claim 1, wherein cosmetic preparation a) consists of comprises at least about 80 wt % of components a1) and a2) relative to its total weight.

8. A cosmetic composition a) comprising, relative to the total weight thereof,
a1) from about 60 to about 98 wt % of at least one polar solvent;
a2) from about 0.5 to about 20 wt % of at least one copolymer of; and
i) vinylpyrrolidone,
ii) vinyl acetate, and
iii) optionally further monomers;
wherein the cosmetic composition is utilized as a material in a device for flash evaporation.

9. Cosmetic product according to claim 1, wherein the cosmetic product is utilized for acting on keratin-containing fibers with the cosmetic preparation a).

10. Cosmetic product according to claim 1, wherein the cosmetic product is utilized for the temporary reshaping of keratin-containing fibers.

11. Method for the temporary reshaping of keratin-containing fibers, the method comprising applying a cosmetic composition a) to the keratin-containing fibers utilizing an apparatus for flash evaporation, the cosmetic composition a) comprising, relative to the total weight thereof,
a1) from about 60 to about 98 wt % of at least one polar solvent;
a2) from about 0.5 to about 20 wt % of at least one copolymer of;
i) vinylpyrrolidone,
ii) vinyl acetate, and
iii) optionally further monomers;

12. The method according to claim 11, wherein
a partial quantity of the cosmetic preparation a) present in a reservoir, inside which there is a pressure corresponding to ambient pressure, is transferred from the reservoir to a container b1);
then, the communication between the reservoir and container b1) is stopped by a flow control component, by means of which such that the flow of cosmetic preparation a) from the reservoir into vessel b1) can be interrupted;
then, the cosmetic preparation a) in container b1), which is closed off from the environment, is heated by a heater, so that the pressure inside vessel b1) rises to values above ambient pressure; and
then, the container b1) in which a pressure above ambient pressure prevails opens in such manner as to allow a partial quantity of the cosmetic preparation in container b1) to escape from container b1) into the environment relieving the pressure that prevailed in container b1) at the time the container was opened, and thus relaxing.

13. Cosmetic product according to claim 1, wherein the cosmetic preparation a) comprises, relative to the total weight thereof, from about 0.01 to about 4.0 wt % of a cationic surfactant a3).

14. Cosmetic product according to claim 13, wherein the cationic surfactant a3) is selected from the groups of quaternary ammonium compounds, esterquats, amidoamines, and combinations thereof.

15. Cosmetic product according to claim 14, wherein the cationic surfactant a3) is selected from the groups of (C₁₂ to C₁₈)-alkyltrimethylammonium salts and combinations thereof.

16. Cosmetic product according to claim 1, wherein the cosmetic preparation a) comprises, relative to the total weight thereof, about 0.1 to about 20 wt % of a film-forming polymer a4).

17. Cosmetic product according to claim 16, wherein the film-forming polymer a4) is selected from the group of anionic polymers.

18. Cosmetic product according to claim 17, wherein the film-forming polymer a4) is selected from the group of copolymers of methacryloylethyl-N,N-dimethylamine oxide and combinations thereof.

19. Cosmetic product according to claim 17, wherein the film-forming polymer a4) is selected from the group of copolymers of i) N-tert-octylacrylamide, ii) acrylic acid, iii) tert-butylaminoethyl methacrylate iv) and optionally further monomers.

20. Cosmetic product according to claim 1, wherein the cosmetic preparation a) comprises at least about 80 wt % of the components a1) and a2) relative to its total weight.
